(19) United States
(12) Patent Application Publication
(10) Pub. No.: US 2019/0133508 A1
(43) Pub. Date: May 9, 2019

(54) SEALED BLOOD COLLECTION DEVICE FOR CLINICAL BLOOD TESTING

(71) Applicant: Chi Wai Yeung, Hong Kong (HK)

(72) Inventor: Chi Wai Yeung, Hong Kong (HK)

(21) Appl. No.: 15/839,822

(22) Filed: Dec. 12, 2017

(30) Foreign Application Priority Data
Nov. 9, 2017 (CN) 201711098637.9

(51) Int. Cl. A61B 5/15

(52) U.S. Cl. CPC A61B 5/150236; A61B 5/150633; A61B 5/150221

(57) ABSTRACT

The present invention relates to a sealed blood collection device for clinical blood testing, comprises a blood collector head, a blood storage device and a discharge mechanism, the blood storage device comprises a blood storage tank, a blood tank entrance disposed at a bottom of the blood storage tank, an extraction piston disposed inside the blood storage tank and a safety pull ring; an external thread is disposed at the upper side wall of the blood storage tank; the extraction piston comprises a rubber piston disposed at a bottom of the blood storage tank and fits against a chamber thereof, a piston rod connected to an upper part of the rubber piston, and a rotatable barrel disposed at an upper part of the blood storage tank and threaded to the blood storage tank, the rotatable barrel comprises a through hole therein; the piston rod penetrates through the through hole, a top end of the piston rod comprises a ring shaped limiter abut against an end surface of the rotatable barrel; a one way valve is disposed inside the blood tank entrance. The blood collection device of the present invention has a reasonable structure, is convenient to use, has good sealing effect, and can also prevent leaks and stings.

### FIELD OF INVENTION

The present invention relates to the field of blood collection devices, in particular to a sealed blood collection device for clinical blood testing.

### BACKGROUND OF INVENTION

Before clinical blood testing, blood collection has to be done through blood collection needles. Blood collection needle is a device for collecting blood samples during clinical testing, made up of a needle head and a needle rod, the needle head is disposed at the head of the needle rod, a cover sleeve is slidably connected to the needle rod, a return spring is disposed between the cover sleeve and the needle rod, and the initial position of the cover sleeve is located at the needle head and the head of the needle rod. Generally, blood collection and blood discharge uses the same needle head, which leads to a weak sealing effect, and easily causes leaks.

### SUMMARY OF INVENTION

The technical problem to be solved by the present invention is: to overcome the aforementioned problem, a sealed blood collection device for clinical blood testing is provided. The blood collection device of the present invention has a reasonable structure, is convenient to use, has good sealing effect, and can also prevent leaks and stings.

The technical solution used in the present invention to solve the technical problem is: a sealed blood collection device for clinical blood testing, comprises a blood collector head, a blood storage device and a discharge mechanism, the blood storage device comprises a blood storage tank, a blood tank entrance disposed at a bottom of the blood storage tank, an extraction piston disposed inside the blood storage tank and a safety pull ring; an external thread is disposed at the upper side wall of the blood storage tank; the extraction piston comprises a rubber piston disposed at a bottom of the blood storage tank and fits against a chamber thereof, a piston rod connected to an upper part of the rubber piston, and a rotatable barrel disposed at an upper part of the blood storage tank and threaded to the blood storage tank, the rotatable barrel comprises a through hole therein; the piston rod penetrates through the through hole, a top end of the piston rod comprises a ring shaped limiter abut against an end surface of the rotatable barrel;

a one way valve is disposed inside the blood tank entrance;

the discharge mechanism comprises a blood discharge needle penetrated into the piston rod, resilient rubber, a press cap and a first compression spring; a conical opening is disposed at a center of the resilient rubber for insertion of the blood discharge needle; an opening is disposed at a center of the press cap; one end of the blood discharge needle penetrates through the rubber piston and connected to the blood storage tank, another end of the blood discharge needle penetrates through the piston rod and inserted into the conical hole of the resilient rubber; ring shaped limiters are disposed on the press cap and the ring shaped limiter and sleeved to each other; the first compression spring is disposed inside the ring shaped limiter, and sleeved outside the discharge needle and the resilient rubber, the two ends of the first compression spring are fixed to the press cap and the ring shaped limiter respectively; the safety pull ring is sleeved onto an exterior of the ring shaped limiter, the top and bottom end of the safety pull ring is abut against the press cap and the rotatable barrel respectively.

In a preferred embodiment, the blood discharge needle is made of plastic.

In a preferred embodiment, the blood collector head comprises a blood collector cap intersleeved to the blood storage tank, a blood collector needle head disposed on the blood tank entrance, and a second compression spring disposed between the blood collector cap and the blood storage tank; an opening is disposed at a center of the blood collector cap for passing of the blood collector needle head.

In a preferred embodiment, the blood collector head comprises a blood collector needle and a blood collector needle head disposed at the blood tank entrance; the two ends of the blood collector needle are connected to the blood collector needle head and the blood tank entrance respectively.

The advantage of the present invention is: the sealed blood collection device for clinical blood testing of the present invention has a reasonable structure, performs one way blood collection, the blood collector head collects blood through pressing of press cap or through blood collector needle. Blood discharge needle is disposed inside resilient rubber, has good sealing effect. When discharging, the safety pull ring is removed, the press cap abuts against the blood storage device, the blood discharge needle penetrates through the resilient rubber, the extracting piston presses downwards, developing a negative pressure at the blood storage tank to complete blood discharging procedure. The blood collection is one way, prevents leaks, the resilient rubber covers the blood discharge needle therefore prevents stings, therefore is safe to use.

### BRIEF DESCRIPTION OF DRAWINGS

The present invention is further explained below with the accompanying drawings and embodiments.

FIG. 1 is a structural schematic diagram of embodiment 1 of a sealed blood collection device for clinical blood testing according to the present invention.

FIG. 2 is a structural schematic diagram of embodiment 2 of a sealed blood collection device for clinical blood testing.

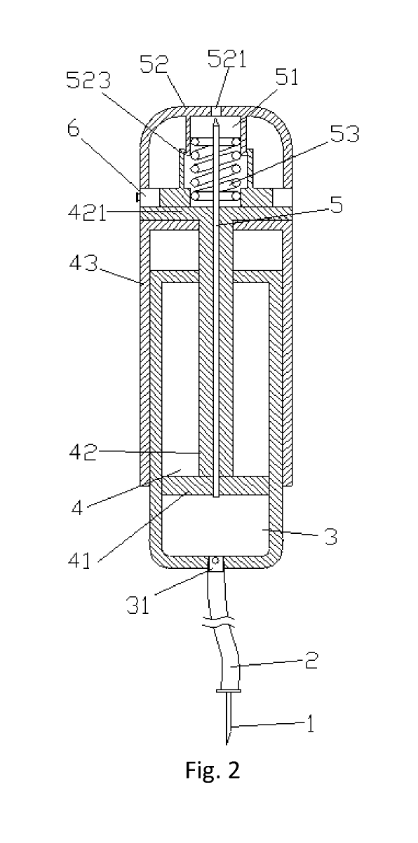

Reference numbers in the drawings: 1 blood collector head, 11 blood collector cap, 111 opening, 12 blood collector needle head, 13 second compression spring, 2 blood collector needle, 3 blood storage tank, 31 blood tank entrance, 4 extraction piston, 41 rubber piston, 42 piston rod, 421 ring shaped limiter, 43 rotatable barrel, 5 blood discharge needle, 51 resilient rubber, 52 press cap, 521 opening, 523 ring shaped limiter, 53 first compression spring, 6 safety pull ring.

### DETAILED DESCRIPTION OF INVENTION

The present invention is further explained below along with the accompanying drawings. The drawings are all simplified schematic diagrams which only illustrates the basic structure of the present invention, as such only the components material to the present invention are shown.

### Embodiment 1

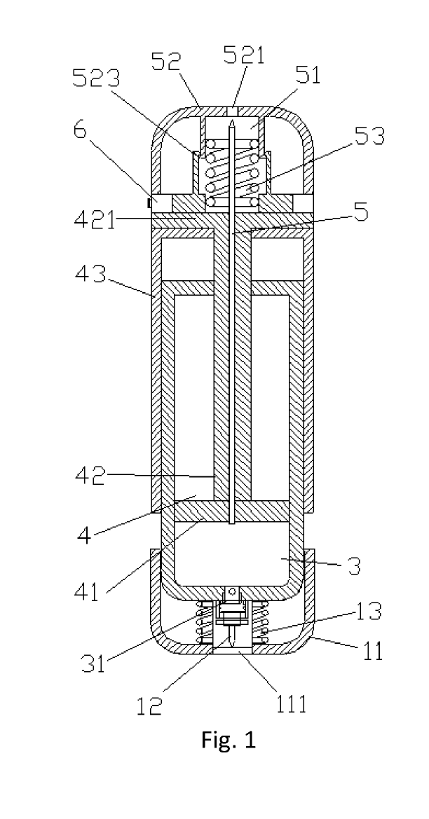

A sealed blood collection device for clinical blood testing as shown in FIG. 1 comprises a blood collector head 1, a blood storage device and a discharge mechanism, the blood storage device comprises a blood storage tank 3, a blood tank entrance disposed at a bottom of the blood storage tank 3, an extraction piston disposed inside the blood storage tank 3 and a safety pull ring 6; an external thread is disposed at the upper side wall of the blood storage tank 3; the extraction piston 4 comprises a rubber piston 41 disposed at a bottom of the blood storage tank 3 and fits against a chamber thereof, a piston rod 42 connected to an upper part of the rubber piston 41, and a rotatable barrel 43 disposed at an upper part of the blood storage tank 3 and threaded to the blood storage tank 3, the rotatable barrel 43 comprises a through hole therein; the piston rod 42 penetrates through the through hole, a top end of the piston rod 42 comprises a ring shaped limiter 421 abut against an end surface of the rotatable barrel 43; a one way valve is disposed inside the blood tank entrance 31; the discharge mechanism comprises a blood discharge needle 51 penetrated into the piston rod 42, resilient rubber 51, a press cap 52 and a first compression spring 53; a conical opening is disposed at a center of the resilient rubber 51 for insertion of the blood discharge needle 5; an opening 521 is disposed at a center of the press cap 52; one end of the blood discharge needle 5 penetrates through the rubber piston 41 and connected to the blood storage tank 3, another end of the blood discharge needle 5 penetrates through the piston rod 42 and inserted into the conical hole of the resilient rubber 51; ring shaped limiters 523 are disposed on the press cap 52 and the ring shaped limiter 421 and sleeved to each other; the first compression spring 53 is disposed inside the ring shaped limiter 523, and sleeved outside the discharge needle 5 and the resilient rubber 51, the two ends of the first compression spring 53 are fixed to the press cap 52 and the ring shaped limiter 421 respectively; the safety pull ring 6 is sleeved onto an exterior of the ring shaped limiter 421, the top and bottom end of the safety pull ring 6 is abut against the press cap 52 and the rotatable barrel 43 respectively.

The blood discharge needle 5 is made of plastic. The blood collector head 1 comprises a blood collector cap 11 intersleeved to the blood storage tank 3, a blood collector needle head 12 disposed on the blood tank entrance 31, and a second compression spring 13 disposed between the blood collector cap 11 and the blood storage tank 3; an opening 111 is disposed at a center of the blood collector cap 11 for passing of the blood collector needle head 12.

### Embodiment 2

A sealed blood collection device for clinical blood testing as shown in FIG. 1, the blood collector head 1 comprises a blood collector needle 2 and a blood collector needle head 12 disposed at the blood tank entrance 31; the two ends of the blood collector needle 2 are connected to the blood collector needle head 12 and the blood tank entrance 31 respectively.

The sealed blood collection device for clinical blood testing of the present invention has a reasonable structure, performs one way blood collection, the blood collector head collects blood through pressing of press cap or through blood collector needle. Blood discharge needle is disposed inside resilient rubber, has good sealing effect. When discharging, the safety pull ring is removed, the press cap abuts against the blood storage device, the blood discharge needle penetrates through the resilient rubber, the extracting piston presses downwards, developing a negative pressure at the blood storage tank to complete blood discharging procedure. The blood collection is one way, prevents leaks, the resilient rubber covers the blood discharge needle therefore prevents stings, therefore is safe to use.

As revealed by the preferred embodiments of the present invention, based on the aforementioned description, a person skilled in the art can make various modifications and adjustments without departing from the technical concept of the present invention. The technical scope of the present invention is not limited to the specification but is defined by the scope of the claims.

What is claimed is:

1. A sealed blood collection device for clinical blood testing, comprises a blood collector head, a blood storage device and a discharge mechanism, the blood storage device comprises a blood storage tank, a blood tank entrance disposed at a bottom of the blood storage tank, an extraction piston disposed inside the blood storage tank and a safety pull ring; an external thread is disposed at the upper side wall of the blood storage tank; the extraction piston comprises a rubber piston disposed at a bottom of the blood storage tank and fits against a chamber thereof, a piston rod connected to an upper part of the rubber piston, and a rotatable barrel disposed at an upper part of the blood storage tank and threaded to the blood storage tank, the rotatable barrel comprises a through hole therein; the piston rod penetrates through the through hole, a top end of the piston rod comprises a ring shaped limiter abut against an end surface of the rotatable barrel;
a one way valve is disposed inside the blood tank entrance;
the discharge mechanism comprises a blood discharge needle penetrated into the piston rod, resilient rubber, a press cap and a first compression spring; a conical opening is disposed at a center of the resilient rubber for insertion of the blood discharge needle; an opening is disposed at a center of the press cap; one end of the blood discharge needle penetrates through the rubber piston and connected to the blood storage tank, another end of the blood discharge needle penetrates through the piston rod and inserted into the conical hole of the resilient rubber; ring shaped limiters are disposed on the press cap and the ring shaped limiter and sleeved to each other; the first compression spring is disposed inside the ring shaped limiter, and sleeved outside the discharge needle and the resilient rubber, the two ends of the first compression spring are fixed to the press cap and the ring shaped limiter respectively; the safety pull ring is sleeved onto an exterior of the ring shaped limiter, the top and bottom end of the safety pull ring is abut against the press cap and the rotatable barrel respectively.

2. The sealed blood collection device for clinical blood testing according to claim 1, characterized in that: the blood discharge needle is made of plastic.

3. The sealed blood collection device for clinical blood testing according to claim 1, characterized in that: the blood collector head comprises a blood collector cap intersleeved to the blood storage tank, a blood collector needle head disposed on the blood tank entrance, and a second compression spring disposed between the blood collector cap and the blood storage tank; an opening is disposed at a center of the blood collector cap for passing of the blood collector needle head.

4. The sealed blood collection device for clinical blood testing according to claim 1, characterized in that: the blood collector head comprises a blood collector needle and a blood collector needle head disposed at the blood tank entrance; the two ends of the blood collector needle are connected to the blood collector needle head and the blood tank entrance respectively.
